SEQUENCE LISTING (10 sequences)

SEQ ID NO: 1 - DNA, 21 bp, Artificial sequence
  PCR primer
gtggagcagcaagagcaagga

SEQ ID NO: 2 - DNA, 21 bp, Artificial sequence
  PCR primer
cttgccccacttaccagtgtg

SEQ ID NO: 3 - DNA, 20 bp, Artificial sequence
  PCR primer
cacgctacaccctggacttg

SEQ ID NO: 4 - DNA, 21 bp, Artificial sequence
  PCR primer
ccatctcctcagcgaagcagt

SEQ ID NO: 5 - DNA, 25 bp, Artificial sequence
  PCR primer
tggcaccacaccttctacaatgagc

SEQ ID NO: 6 - DNA, 25 bp, Artificial sequence
  PCR primer
gcacagcttctccttaatgtcacgc

SEQ ID NO: 7 - DNA, 20 bp, Artificial sequence
  PCR primer
acgtggctaagaatgtcatc

SEQ ID NO: 8 - DNA, 11 bp, Artificial sequence
  PCR primer
ctggtaggcga

SEQ ID NO: 9 - DNA, 20 bp, artificial sequence
  PCR primer
atgagggcctggatcttctt

SEQ ID NO: 10 - DNA, 20 bp, Artificial sequence
  PCR primer
gcttctgcttctgagtcaga

(19) United States
(12) Patent Application Publication
(10) Pub. No.: US 2005/0074877 A1
(43) Pub. Date: Apr. 7, 2005

(54) Biological engineering of articular structures containing both cartilage and bone

(76) Inventor: Jeremy Jian Mao, Chicago, IL (US)

Correspondence Address: WELSH & KATZ, LTD, 120 S RIVERSIDE PLAZA, 22ND FLOOR, CHICAGO, IL, 60606, US

(21) Appl. No.: 10/899,964

(22) Filed: Jul. 27, 2004

Related U.S. Application Data
(60) Provisional application No. 60/490,640, filed on Jul. 28, 2003.

(51) Int. Cl. C12N 5/08

(52) U.S. Cl. 435/366; 435/372

(57) ABSTRACT

De novo organogenesis of a joint or portion thereof by osteochondral constructs comprising adult mesenchymal stem cells (MSCs) encapsulated on a scaffold is disclosed. MSCs-derived chondrogenic and osteogenic cells can be loaded in hydrogel monomer suspensions in distinct stratified and yet integrated layers that are sequentially photopolymerized in a mold. Constructs can be then implanted in vivo in a host and fabricated therein or, alternatively, the constructs can be incubated ex vivo, both procedures producing a functional joint or portion thereof.

### CROSS-REFERENCE TO RELATED APPLICATION

This application is based on Ser. No. 60/490,640 filed on Jul. 28, 2003.

### GOVERNMENTAL SUPPORT

The present invention was made with governmental support pursuant to NIH grants DE13964 and DE13088. The government has certain rights I the invention.

### TECHNICAL FIELD

This invention relates to the biological engineering of bone and cartilage de novo. More particularly, this invention relates to stem cell-driven organogenesis of functionalized synovial joints.

### BACKGROUND OF THE INVENTION

All skeletal motion in terrestrial mammals is made possible by the operation of synovial joints. The architecture of a synovial joint is intriguing in that cartilage and bone, two distinct adult tissue phenotypes with little in common, structurally integrate and function in synchrony allowing flexible limb movement yet can withstand mechanical loading up to several times body weight [Martin, R. B., et al. Skeletal Tissue Mechanics Springer-Verlag, New York, 79-126, (1998)].

With age, trauma, and physical activity, the cartilage and bone structures of synovial joints can deteriorate, resulting in debilitating ailments such as osteoarthritis, rheumatoid arthritis, ankylosis, dysfunctional syndromes, and bone fractures. These ailments necessitate billion-dollar expenditures in medical care and rehabilitation. [Gravallese, E. M. Ann. Rheum. Dis. 61:84-86 (2002)]. For example, osteoarthritis (OA) alone aggravates millions of individuals as nearly every person aged 65 and older becomes afflicted. [Hayes, D. W., Jr. et al. Clin. Podiatr. Med. Surg. 18:35-53 (2001)].

This epidemic of synovial joint disorders has motivated numerous studies aiming to improve the quality of life and medical care for affected patients. However, for most of these patients, surgical total joint replacement is the only clinical option. [Buckwalter, J. A. Clin. Orthop. 402:21-37 (2002)]. Typical total joint replacement surgery consists of the removal of the damaged joint or parts followed by the implantation of a metal prosthesis in the shape of the bone fitted into a polyethylene socket. Other less common options include autografts, allografts, and xenografts to name a few. [Bugbee, W. D. J. Knee Surg. 15:191-195 (2002)]. Even less common are cartilage cell transfer and cartilage plug transplantation (mosaicplasty). At the present time, current treatment approaches are plagued by problems such as donor site morbidity, limited tissue supply, immunorejection, potential transmission of pathogens, implant loosening, and mechanical breakdown. [Hangody, L. et al. Clin. Orthop. S391:S328-336 (2001)].

The biological engineering of joint components is in its infancy. Cartilage regeneration by means of isolated chondrocytes or mesenchymal stem cells (MSCs) or resurfacing of surgically created cylindral articular defects has shown encouraging results in animal models. [Bentley, G. et al. Biomed. Mater. Res. 28:891-899 (1994); Goldberg, V. M. & Caplan, A. I. Orthopedics 17:819-821 (1994); Vacanti, C. A. et al. Am. J. Sports Med. 22:485-488 (1994)]. Bone regeneration by encapsulating MSCs or growth factors in polymer scaffolds has shown considerable promise toward tissue-engineered repair of bony defects. [Bruder, S. P. et al. Clin. Orthop. 355:S247-256 (1998); Hollinger, J. O. et al. J. Biomed. Mater. Res. 43:356-364 (1998); Winn, S. R. et al. J. Biomed. Mater. Res. 45:414-421 (1999); Sikavitsas, V. I. et al. J. Biomed. Mater. Res. 62:136-148 (2002)].

In contemplating the biological engineering of a synovial joint, the structural characteristics of the joint must be considered. The most prominent feature of a synovial joint is the condyle, the protuberant portion similar to the knuckle, which consists of a thin layer of cartilage residing over bone structure [Martin, R. B. et al. Skeletal Tissue Mechanics, Springer Verlag, New York, pp. 79-126, (1998)]. Cartilage consists of mature cartilage cells (chondrocytes) embedded in a hydrated extracellular matrix [Mow, V. C. and Hayes, W. C. Basic Orthopaedic Biomechanics, New York, Raven Press, pp. 143-199, (1991)]. Chondrocytes are crucial to cartilage histogenesis and maintenance [Hunziker, E. B. Osteoarth. Cartil. 10:432-463 (2002)]. Mature cartilage only has a limited number of resident chondrocytes [Volk, S. W. and Leboy, P. S. J. Bone Miner. Res. 14:483-486 (1999)]. Although all cartilage cells are called chondrocytes, they represent a heterogeneous group of cells, the majority of which are differentiated chondrocytes rather than cartilage-forming chondroblastic cells or their progenitors, mesenchymal stem cells (MSCs) [Pacifici, M. et al. Conn. Tis. Res. 41:175-184 (2000)]. Thus, few chondrocytes are available for regeneration upon cartilage injuries at the injured site [Hunziker, E. B. Osteoarth. Cartil. 10:432-463 (2002)].

However, there is overwhelming evidence that adult bone marrow contains MSCs that can differentiate into virtually all lineages of connective tissue cells such as osteogenic, chondrogenic, tenocytes, adipogenic, odontoblastic, etc. [Goldberg, V. M. and Caplan, A. I. Orthopedics 17:819-821 (1994)]. The MSCs' role in fracture healing which includes multiple phenotypic switches between fibrous, hyaline cartilage, fibrocartilage, and bone further indicates their multipotent nature [Einhorn, T. A. Clin. Orthop. 355 Suppl.:S7-S21, (1998)]. The techniques of harvesting and culturing MSCs from tibiofemoral bone marrow as well as inducing MSCs to differentiate into chondrogenic and osteogenic cell lineages in vitro and in vivo have been successful. [Alhadlaq, et al. Ann. Biomed. Eng. 32:911-923, (2004)].

In addition to the cartilage, another crucial joint part is the bone structure. Bone represents a different connective tissue phenotype from cartilage despite the fact that cartilage and bone both derive from MSCS. [Caplan, A. I. J. Orthop. Res. 9:641-649 (1991)]. Subchondral bone is rich in blood supply and is organized into trabeculae, each consisting of islands of mineralized collagen matrix with osteoblasts residing on the trabecular surface with osteocytes embedded in the mineralized matrix [Buckwalter, J. A. Clin. Orthop. 402:21-37 (2002)]. During normal development, hypertrophic chondrocytes in articular cartilage undergo apoptosis followed by degeneration of their matrices and the invasion of osteogenic cells with angiogenesis [Volk, S. W., and Leboy, P. S. J. Bone Miner. Res. 14:483-486 (1999)].

As such, both soft and hard scaffolds have been used for bone engineering. Hard scaffold materials, such as hydroxyapatite, can provide stiff mechanical support, whereas soft polymers, such as hydrogels, permit more homogenous cell seeding and room for the formation of bone matrix in vivo [Bruder, S. P. et al. Clin. Orthop. 355 Suppl:S247-S256, (1998)].

Also to be considered in cartilage regeneration and/or de novo formation is the importance of biocompatible polymers. It is known that 95% of cartilage volume is extracellular matrix [Mow, V. C., and Hayes, W. C. Basic Orthopaedic Biomechanics, New York, Raven Press, pp. 143-199, (1991)] consisting of collagen framework residing within hydrated proteoglycan macromolecules [Pacifici, M., et al. Conn. Tis. Res. 41:175-184 (2000)]. Cartilage proteoglycans are negatively charged molecules that retain abundant water molecules.

A mimic of a cartilage proteoglycan is a hydrogel, a hydrophilic polymer capable of absorbing biological fluids while maintaining a three-dimensional structure. [Lee, K. Y., and Mooney, D. J. Chem. Rev. 101:869-879 (2001)]. Hydrogel scaffolds can provide tissue-forming cells, such as chondrocytes, with a mimicked environment of the extracellular matrix. [Oxley, H. R. et al. Biomaterials 14:1064-1072 (1993)]. A large number of hydrogel polymers have been widely utilized in cartilage tissue engineering including alginate, polylactic acid (PLA), polyglycolic acid (PGA) or their copolymer (PLGA), chitosan, and poly-ethylene glycol-based polymers (PEG) [Lee, K. Y., and Mooney, D. J. Chem. Rev. 101:869-879 (2001)].

Although a few animal models have demonstrated some success in repairing small joint defects through tissue engineering, many problems still persist. The pending problems are a lack of use of adult stem cells [Poshusta, A. K. & Anseth, K. S. Cells Tissues Organs 169:272-278 (2001)], a lack of definitive shape formation of the articular condyle [Lennon, D. P. et al. Exp. Cell Res. 219:211-222 (1995)], a lack of use of both the cartilage and bone components [Abukawa, H. et al. J. Oral Maxillofac. Surg. 61:94-100 (2003)]. Another technique, mosaicplasty, can be applied toward larger size defects by harvesting multiple plugs of osteochondral cylinders from non-load bearing regions of the articular condyle and transplanting to load-bearing regions [Bugbee W. D. J. Knee Surg. 15:191-195, (2002)]. Although multiple plugs can be applied to repair larger size defects, mosaicplasty necessitates donor site defects and is limited by the availability of healthy unloaded joint regions.

Other efforts to reconstruct condyles have focused on the fabrication of chondral or osteochondral constructs by harvesting chondrocytes from the mandibular or appendicular joints or osteoblasts of the calvaria and periosteum [Poshusta A. K. and Anseth K. S. Cells Tissues Organs 169:272-278 (2001)]. The problem with these approaches is the fact that the seeded cells are articular chondrocytes (e.g., one cannot harvest articular chondrocytes by sacrificing the patient's elbow joint to tissue-engineer his/her knee joint). This rules out their ultimate applications in autologous reconstruction of the human articular condyle.

Moreover, no effort has been made to mimic natural cartilage development by creating stratified chondrogenic layers of tissue-engineered articular condyle. Distinct chondrocyte layers are necessary for orchestrated progression of normal cartilage development [Pacifici, M., et al. Conn. Tis. Res. 41:175-184 (2000)].

Lastly, little progress has been made to couple mechanical stimulation of cell-polymer constructs with their in vivo regenerative outcome. Mechanical stresses readily modulate cell differentiation and matrix synthesis of not only natural bone and cartilage, but also fabricated chondral constructs. For example, there is overwhelming evidence at various levels of organization that cartilage development and health are modulated by mechanical stresses [Kantomaa, T., and Hall, B. K. J. Anat. 161:195-201 (1988)].

There is also evidence that mechanical stresses readily modulate the proliferation, differentiation, and matrix synthesis of bone cells [Rubin, J., Crit. Rev. Eukaryot. Gene Expr. 5:177-191 (1995)]. As another example, chondrocytes seeded in agarose disks subjected to 3 percent dynamic strain at 0.01 Hz-1 Hz increase biosynthetic activity. [Buschmann, M. D. et al. J. Cell Sci. 108:1497-1508, (1995)]. Agarose-encapsulated chondrocytes harvested from superficial and deep zones of articular cartilage respond differently to dynamic compression with increased GAG synthesis by deep cells but decreased GAG synthesis by superficial cells and increasing proliferation [Lee, K. Y., and Mooney, D. J. Chem. Rev. 101:869-879, (2001)]. Dynamic compression at 1 Hz and 10 percent strain increases equilibrium modulus over controls, from 15 kPa to 100 kPa, as well as GAG and hydroxyproline content [Mauck, R. L. et al. J. Biomech. Eng. 122:252-260 (2000)].

Moreover, intermittent stresses increase both collagen and GAG contents synthesized by immature and adult chondrocytes seeded in PGA meshes [Carver, S. E., and Heath, C. A. Biotechnol. Bioeng. 62:166-174 (1999)]. Chondrocytes seeded in PGA scaffolds and cultured in a rotating wall bioreactor showed superior mechanical properties and biochemical compositions to static flask culture [Vunjak-Novakovic, G. et al. J. Orthop. Res. 17:130-138 (1999)]. Dynamic compression at 5 percent strain had stimulatory effects on synthesis that were dependent on the static offset compression amplitude (10 percent or 50 percent) and dynamic compression frequency (0.001 or 0.1 Hz) [Davisson, T. et al. J. Orthop. Res. 20:842-848 (2002)].

Further, bovine calf chondrocytes seeded in benzylated hyaluronan and polyglycolic acid with sponge, non-woven mesh, and composite woven/non-woven mesh upon treatment in bioreactor demonstrated different cell densities and matrix syntheses such as GAG, total collagen, and type-specific collagen mRNA expression [Pei, M., et al. FASEB J. 16:1691-1694, (2002)]. Moreover, static compression decreased protein and proteoglycan biosynthesis in a time- and dose-dependent manner, whereas selected dynamic compression protocols were able to increase rates of collagen biosynthesis [Lee, C. R. et al. J. Biomed. Mater. Res. 64A:560-569 (2003)]. Also, bovine articular chondrocytes seeded in porous collagen sponges subjected to constant or cyclic (0.015 Hz) fluid compression at 2.8 MPa demonstrated increased GAG content [M]

The present invention, as disclosed hereinafter, provides biologically engineered joints derived from stem cells and a biocompatible scaffold. This invention can benefit the many millions of patients who suffer from osteoarthritis, rheumatoid arthritis, bone or cartilage injuries, and congenital anomalies.

### BRIEF SUMMARY OF THE INVENTION

The present invention provides a synthetic partial or entire joint in human form prepared in vivo or ex vivo (de novo) by growing stem cells such as embryonic stem cells or adult stem cells derived from bone marrow, adipose tissue, peripheral blood or other tissue on a biocompatible scaffold. The preferred biocompatible scaffold is comprised of polymerized (polyethylene glycol) diacrylate. Another embodiment of the present invention is an osteochondral construct from which a joint is fabricated that comprises a biocompatible scaffold and at least two types of stem cells, preferably adult mesenchymal stem cells, wherein a first cell type is differentiated into a chondrocyte and the second cell type is differentiated into an osteoblast.

Another embodiment is a method of producing an osteochondral construct comprising the steps of providing stem cells such as those from bone marrow, adipose tissue, peripheral blood or the like. Treating a first portion of the cells with a chondrogenic medium to induce differentiation into chondrocytes, and treating a second portion of the cells with an osteogenic medium to induce differentiation into osteoblasts. The chondrocytes and osteoblasts are loaded into a biocompatible scaffold, and the scaffold-containing chondrocytes and osteoblasts is then maintained under biological growth conditions for a time period sufficient for the osteoblasts and chondrocytes to grow. Still another embodiment is a method of producing a biologically engineered joint by either an in vivo implantation of an osteochondral construct into a host animal or an ex vivo incubation of an osteochondral construct in a chamber.

The present invention has several benefits and advantages. One benefit is that a truly biologically engineered joint can overcome deficiencies associated with current cartilage/bone grafts and artificial prostheses and is capable of remodeling during physiological function, thus mimicking normal joints. Still further benefits and advantages of the invention will be apparent to those skilled in this art from the detailed description that follows.

### BRIEF DESCRIPTION OF THE DRAWINGS

In the drawings forming a part of this invention,

FIG. 1 is a series of photographs that illustrate the fabrication of a human-shaped articular condyle from rat bone marrow-derived mesenchymal stem cells (MSCs). FIG. 1A shows the recovery of a tissue-engineered articular condyle after 4-week subcutaneous implantation of the osteochondral construct in the dorsum of immunodeficient mice. FIG. 1B shows a top view of the recovered osteochondral construct that retained the shape of the molded articular condyle. Transparent and photo-opaque portions of the construct represent cartilaginous and osseous components of the tissue-engineered articular condyle as evidenced by histologic characteristics of the chondral and osseous components in FIG. 2. FIG. 1C shows an acrylic model made from an alginate impression of a human cadaver mandibular condyle. FIGS. 1D and 1E are photographs that show that a polyurethane negative mold of FIG. 1C and fits the acrylic human articular condyle model. FIG. 1F is a photograph of a human-shaped mandibular condyle construct fabricated in a two-phase process in which: 1) photopolymerizable PEG-hydrogel monomers encapsulating MSC-derived chondrogenic cells was loaded to occupy the top 2 mm of the negative model (above the thin line in 1F) followed by photopolymerization; and 2) additional further photpolymerizable PEG-hydrogel monomers encapsulating MSC-derived osteogenic cells loaded to occupy the 4 mm space below the thin line in FIG. 1F followed by photopolymerization. Thus, PEG-based hydrogels above and below the red line in FIG. 1F were fully integrated as evidenced in FIG. 2A, below. The dimensions are shown in millimeters by the ruler at the bottom of the figure.

FIG. 2 contains photomicrographs of a tissue-engineered articular condyle recovered after 4 weeks of in vivo implantation. FIG. 2A show HE stained section of the osteochondral interface showing full integration of the PEG hydrogel encapsulating MSC-derived chondrogenic and osteogenic cells photopolymerized in a two-phase process. The left half of FIG. 2A shows the chondrogenic portion characterized by abundant intercellular space between MSC-derived chondrocyte-like cells. The right half of FIG. 2A shows the osteogenic portion characterized by intercellular mineralization nodules that were confirmed to be mineral crystals by von Kossa staining (Sigma Cat # S-8157, N-8002, T-0388, A-7210). FIG. 2B shows the presence of cartilage-specific glycosaminoglycans not only in the pericellular zones, but also the intercellular matrix as evidenced by positive safranin O red stain. FIG. 2C is a HE stained section showing a representative island of trabecula-like bone structure with MSC-derived osteoblast-like cells. FIG. 1D shows trabecula-like structures positively stained by toluidine blue that indicates osseous tissue formation. Dimension bars indicate the relative sizes of the depicted structures.

FIG. 3 illustrates mesenchymal stem cells (MSCs) induced to differentiate into chondrogenic and osteogenic cells ex vivo. FIG. 3A shows a primary MSCs culture-expanded for 2 weeks adhered to culture plate. FIG. 3B shows a Nomarski contrast image [Kouri J B, et al. Microsc Res Tech. 1998 Jan. 1; 40(1):22-36. Review] of two MSCs cultured on glass cover slip, showing typical spindle shape. FIG. 3C illustrates ex vivo fabrication of a bilayered osteochondral construct incubated for 6 weeks showing layer-specific localization of MSC-derived chondrogenic and osteogenic cells without migration across the interface, in corroboration with in vivo findings shown in FIG. 2A. FIG. 3D shows a live/dead cell labeling study that verified that the majority of MSCs survived photopolymerization. Live cells are labeled green with calcein. FIG. 3E shows a representative force curve generated during nanoindentation of PEG hydrogel with atomic force microscopy (AFM) that illustrates nanoscale adhesive forces upon the AFM scanning tip approaching and retracting from the sample surface. FIG. 3F shows a representative force curve upon nanoindentation of PEG-hydrogel encapsulating MSC-derived chondrogenic cells after 4-week incubation. Note that nanoindentation forces were approximately two fold higher than PEG-hydrogel alone shown in FIG. 3E. FIG. 3G shows a representative force curve upon nanoindentation of PEG-hydrogel encapsulating MSC-derived osteogenic cells after 4-week incubation. Note that nanoindentation forces were much higher than in PEG-hydrogel alone shown in FIG. 3E. FIG. 3H shows the mean Young's modulus of the osteogenic PEG hydrogel (N=8) was significantly higher than the chondrogenic PEG hydrogel (N=12), both of which were significantly higher than PEG-hydrogel without cells (N=9).

FIG. 4 shows a series of photomicrographs and results from TGF-β1-mediated, MSC-derived chondrogenesis in monolayer culture and after encapsulation in PEG-hydrogel. FIG. 4A shows positive safranin-O reaction of MSC-derived chondrogenic cells after 4-week monolayer culture. FIG. 4B shows MSC-derived chondrogenic cells which were encapsulated in PEG hydrogel incubated in chondrogenic medium for 4 weeks also showed positive safranin-O staining. FIG. 4C is a gel illustrating that RNA extracted from PEG hydrogels encapsulating MSC-derived chondrogenic cells showed upregulated expression of aggrecan and Type II collagen compared to RNA from gels incubated without TGF-β1. Lane 1: MSC in DMEM (10% FBS) monolayer culture; Lane 2: MSC cultured in chondrogenic medium with TGF-β1 for 3 weeks; Lane 3: MSC cultured in chondrogenic medium with TGF-β1 for 6 weeks; Lane 4: MSC cultured in chondrogenic medium in absence of TGF-β1 for 6 weeks; FIG. 4D and FIG. 4E show chondrogenesis indicated by increases in total glycosaminoglycan (GAG) content (FIG. 4D) and total collagen content (FIG. 4E) in PEG hydrogel encapsulating MSC-derived chondrogenic cells following 0, 3 and 6 weeks of incubation in chondrogenic medium containing TGF-β1.

FIG. 5 illustrates MSC-driven osteogenesis in monolayer culture and after encapsulation in PEG-hydrogel upon induction by osteogenic medium containing dexamethasone, β-glycerophosphate, and ascorbic acid. FIG. 5A shows the positive reaction of MSC monolayer culture to alkaline phosphatase (arrow) and von Kossa silver (arrow) after 4 week treatment in osteogenic medium. FIG. 5B shows matrix mineral deposition in PEG hydrogel encapsulating MSC-derived osteogenic cells (von Kossa silver staining). FIG. 5C shows a gel with increasing RNA expression of osteonectin and alkaline phosphatase over time (Lane 1: 1-week incubation; Lane 2: 3-week incubation; Lane 3: 6-week incubation). FIG. 5D shows increasing calcium content in PEG hydrogel encapsulating MSC-derived osteogenic cells up to 6 weeks in incubation in osteogenic medium.

FIG. 6 is diagram of the experimental protocol followed in the preparation of a biologically engineered joint. A: Harvest of mesenchymal stem cells (MSCs) from the rat tibiofemoral complex. B: Primary MSC culture-expansion. C: Treatment of a single population of expanded MSCs with chondrogenic medium containing TGF-β1 (one portion of cells), and osteogenic medium containing dexamethasone, β-glycerophosphate, and ascorbic acid (remaining portion of cells). D: Preparation of PEG-hydrogel suspensions of MSC-derived chondrogenic and osteogenic cells. E: Loading PEG-hydrogel suspensions with MSC-derived chondrogenic cells in lower layer of the negative mold of the articular condyle (approx. thickness: 2 mm; cf. FIGS 1D and 1F—reversed orientation) followed by F: Photopolymerization with UV light. Next, loading PEG-hydrogel suspension with MSC-derived osteogenic cells to occupy the upper layer of the negative mold of the articular condyle, followed by photopolymerization. The fabricated osteochondral constructs (G) were implanted in subcutaneous pockets of the dorsum of immunodeficient mice (H).

### DETAILED DESCRIPTION OF THE INVENTION

The present invention contemplates the biological engineering of bone and cartilage. Specifically, this invention relates to the de novo synthesis of a synovial joint or a portion thereof that is prepared from stem cells, embryonic or adult stem cells, and a biocompatible scaffold. Embryonic and adult stem cells are well known and need not be discussed herein. These cells can be obtained from bone marrow, adipose tissue and peripheral blood, as well as from other sources, as is also well known.

Adult mesenchymal stem cells are preferred and are used illustratively herein with the understanding that fetal stem cells or other adult stem cells can be used. The adult mesenchymal cells are derived from bone marrow cells in which at least one cell has differentiated into an osteoblast and at least one cell has differentiated into a chondrocyte. The biocompatible scaffold preferably is comprised of polymerized (polyethylene glycol) diacrylate. In one embodiment, the joint is fabricated in vivo by the stem cells. In another embodiment, the joint is prepared ex vivo by the stem cells. Most preferably, the joint is fabricated in human form.

Another embodiment of the present invention is directed to an osteochondral construct from which a joint is fabricated. The construct comprises a biocompatible scaffold and stem cells in which at least some of those stem cells are differentiated into chondrocyte cells and some are differentiated into osteoblast cells. The preferred biocompatible scaffold is comprised of polymerized (polyethylene glycol) diacrylate.

A preferred scaffold is in a physically defined form; i.e., a material that maintains its physical form at the temperatures of use. That scaffold can be a gel or rigid, and can be in a shape that is a mesh, powder, sponge, or solid.

Preferably, the scaffold comprises a polymer. A preferred polymeric scaffold comprises a polymer material selected from the group consisting of polylactic acid, polyglycolic acid, polymerized (polyethylene glycol) diacrylate, polymerized (polyethylene glycol) dimethacrylate and mixtures thereof. More preferably, the polymeric scaffold is prepared from a photopolarizable hydrogel monomer. Most preferred is (polyethylene glycol) diacrylate monomer [MW 3400; Shearwater Polymers, Huntsville, Ala.]. A (polyethylene glycol) diacrylate or dimethacrylate monomer can have a molecular weight of about 3400 to about 100,000. In a different embodiment, the scaffold comprises a natural material selected from the group consisting of alginate, chitosan, coral, agarose, fibrin, collagen, bone, silicone, cartilage, hydroxyapatite, calcium phosphate, and mixtures thereof.

Preferably, the construct further comprises an osteogenic agent. In particular, a preferred osteogenic agent include dexamethasone, bone morphogenetic protein (BMP) and transforming growth factor (TGF) beta super families such as BMP2. The construct can also comprise a chondrogenic agent. A preferred chondrogenic agent is a TGFβ1, a member of the transforming growth factor-beta superfamily such as TGF-β1, or a vitamin A analog such as ascorbic acid.

In another embodiment, the present invention comprises a composition in the shape of a partial or entire joint comprising a biocompatible scaffold wherein the scaffold is comprised of a matrix, an osteogenic agent, a chondrogenic agent, a nutrient medium, at least one antibiotic, and at least two types of stem cells, wherein at least one of the cell types is differentiated into a chondrocyte and the other of the cell types is differentiated into an osteoblast. In this embodiment, preferably, the matrix comprises polymerized (polyethylene glycol) diacrylate that has been polymerized by the action of ultraviolet light and a photoinitiator such as 2-hydroxy-1-[4-(hydroxyethoxy)phenyl]-2-methyl-1-propanone (Ciba, Tarrytown, N.Y.).

In yet another embodiment, this invention contemplates a composition in the shape of a partial or entire joint comprising a biocompatible scaffold wherein the scaffold is comprised of polymerized (polyethylene glycol) diacrylate, 2-hydroxy-1-[4-(hydroxyethoxy)-phenyl]-2-methyl-1-propanone (a biocompatible photoinitiator), dexamethasone, transforming growth factor beta-1, a nutrient medium comprising beta-glycerophosphate and ascorbic acid 2-phosphate, penicillin, streptomycin, and at least two types of stem cells, such as adult mesenchymal stem cells derived from human bone marrow, wherein at least one of the cell type is differentiated into a chondrocyte, and the other cell type is differentiated into an osteoblast.

The present invention also encompasses a composition in the shape of a partial or entire joint comprising a biocompatible scaffold wherein the scaffold is comprised of polymerized (polyethylene glycol) diacrylate, 2-hydroxy-1-[4-(hydroxyethoxy)phenyl]-2-methyl-1-propanone, dexamethasone, transforming growth factor beta-1, a nutrient medium comprising beta-glycerophosphate and ascorbic acid 2-phosphate, penicillin, and streptomycin and stem cells that are differentiated into chondrocytes and stem cells differentiated into osteoblasts. Both cell types are preferably derived from adult mesenchymal stem cells from human bone marrow.

A further embodiment of the present invention contemplates a method of producing an osteochondral construct comprising the steps of harvesting stem cells; treating one portion of the cells with chondrogenic medium to induce differentiation into chondrocytes; treating a further portion of the cells with osteogenic medium to induce differentiation into osteoblasts and loading the chondrocytes and osteoblasts onto a biocompatible scaffold.

The present invention also relates to a method of producing a biologically engineered partial or entire joint in vivo comprising implanting a composition comprising a biocompatible scaffold and at least two types of human stem cells as discussed before into a host. In this embodiment the method preferably comprises subjecting the cells to mechanical stresses conducive to either osteogenesis or chondrogenesis or both.

In another embodiment, the present invention contemplates a method of producing a biologically engineered partial or entire joint ex vivo comprising attaching at least two types of stem cells, as discussed before, to a biocompatible scaffold wherein the scaffold is comprised of a matrix, an osteogenic agent, a chondrogenic agent, a nutrient medium and at least one antibiotic. In this embodiment, preferably, the method comprises subjecting the cells to mechanical stresses conducive to either osteogenesis or chondrogenesis or both.

In yet another embodiment, the present invention contemplates a method of producing a biologically engineered partial or entire joint in vivo comprising the steps of harvesting stem cells, such as adult mesenchymal stem cells (MSCs) from bone marrow; expanding the MSCs; treating a portion of the expanded MSCs with chondrogenic medium containing TGF-β1; treating a second portion of the expanded MSCs with osteogenic medium containing dexamethasone, β-glycerophosphate, and ascorbic acid; creating a PEG-hydrogel monomer suspension of the MSC-derived chondrogenic cells; creating a PEG-hydrogel monomer suspension of the MSC-derived osteogenic cells; loading the PEG-hydrogel monomer suspension of MSC-derived chondrogenic cells in a negative mold of a joint or partial joint; loading the PEG-hydrogel monomer suspension of MSC-derived osteogenic cells in the negative mold of the joint or partial joint; photopolymerizing the PEG-hydrogel monomer suspension with UV light to create a fabricated osteochondral construct; implanting the fabricated osteochondral construct in a host; and harvesting a joint or partial joint prepared from the osteochondral construct.

### EXAMPLE 1

### Organogenesis of Articular Condyles in Vivo

Generic articular condyles, shaped from the negative mold of a cadaver human mandibular condyle, were formed de novo in subcutaneous pockets of the dorsum of immunodeficient mice after in vivo implantation of osteochondral constructs consisting of MSC-derived chondrogenic and osteogenic cells encapsulated in a photochemically-polymerized poly(ethylene glycol)-based hydrogel (PEG hydrogel). Cell-hydrogel constructs were photopolymerized in a two-phase process so that PEG gel-encapsulated chondrogenic cells fully integrated with PEG gel-encapsulated osteogenic cells. Organogenesis of the articular condyles occurred 4 weeks after surgical implantation of these bilayered, condyle-shaped osteochondral constructs in the dorsum of immunodeficient mice.

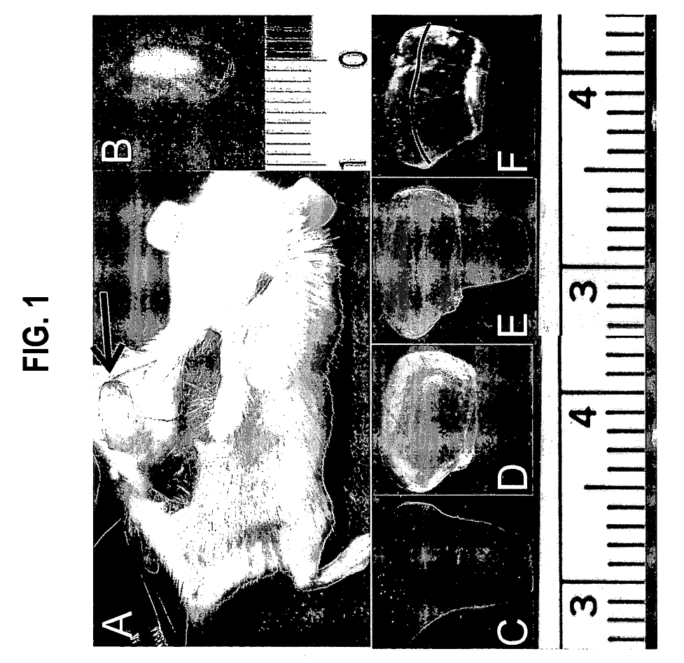

The recovered articular condyles from in vivo implantation (FIGS. 1A and 1B) resembled the macroscopic shape of the cell-hydrogel construct (FIG. 1F) as well as the positive and negative condylar molds (FIGS. 1C and 1D, respectively), which showed a close fit to the fabricated articular condyle before in vivo implantation (FIG. 1E).

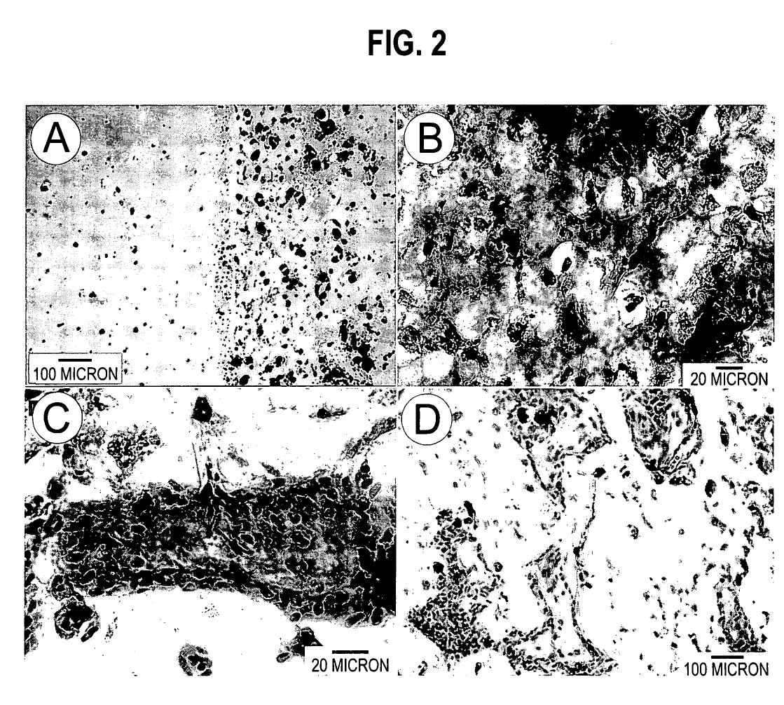

There were both a superficial transparent portion and an inner photo-opaque portion in the superior (top) view of the recovered articular condyle (FIG. 1B), representing chondrogenic and osteogenic elements, respectively, as evidenced below. The interface between the upper-layer PEG hydrogel incorporating MSC-derived chondrogenic cells and the lower-layer incorporating MSC-derived osteogenic cells (cf., above and below the line in FIG. 1F) demonstrated distinctive microscopic characteristics (FIG. 2A).

The chondrogenic layer (the left half of FIG. 2A) contained chondrocyte-like cells surrounded by abundant intercellular matrix. By contrast, the osteogenic layer (the right half of FIG. 2A) contained intercellular mineralization nodules that were confirmed to be mineral crystals by von Kossa staining. The chondrogenic layer showed intense reaction to safranin-O (FIG. 2B), a cationic chondrogenic marker that binds to cartilage-specific glycosaminoglycans such as chondroitin sulfate and keratan sulfate. Some of the MSC-derived chondrogenic cells were surrounded by pericellular matrix, characteristic of natural chondrocytes (FIG. 2B). The osteogenic layer demonstrated multiple islands of bone trabecula-like structures occupied by osteoblast-like cells as exemplified in FIG. 2C that reacted positively to von Kossa silver stain indicating its osteogenic tissue phenotype (FIG. 2D).

### EXAMPLE 2

### Differentiation of MSCs and Stratified PEG Hydrogel Encapsulation

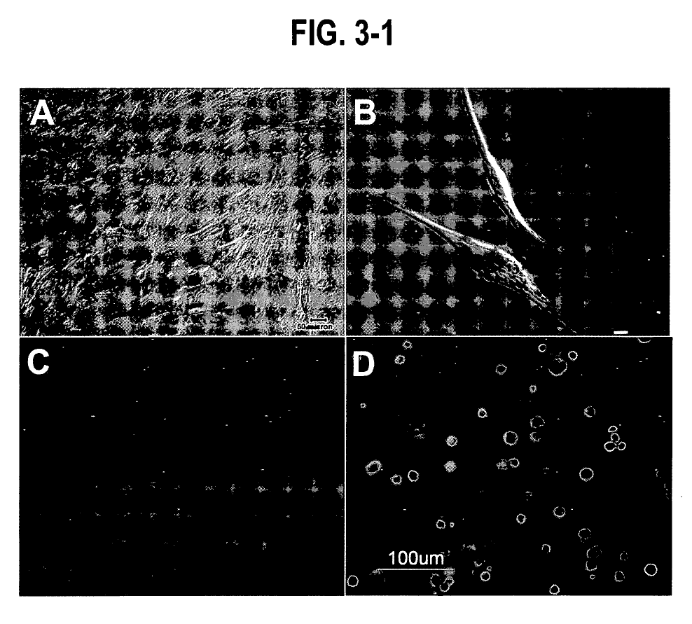

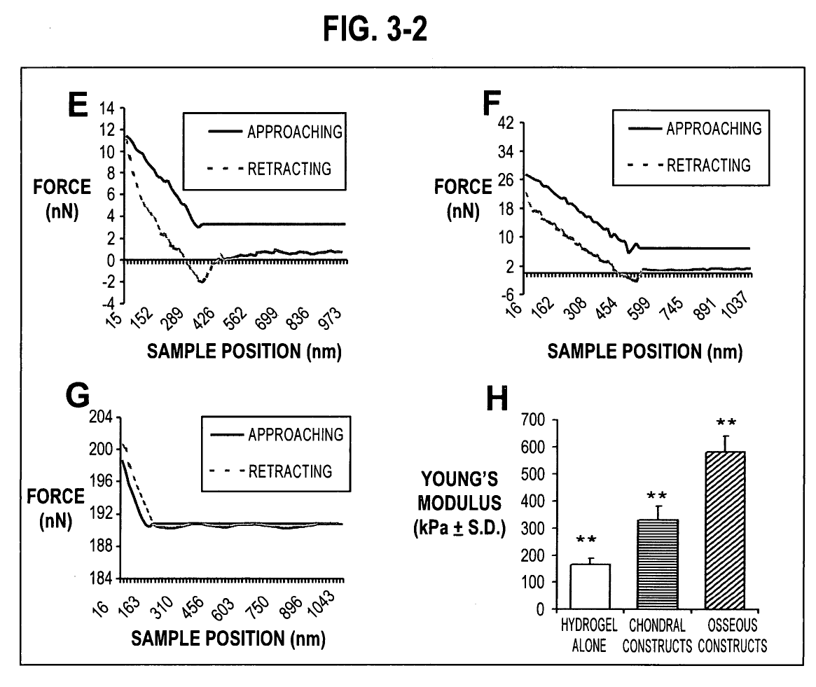

Marrow-derived MSCs adhered to the culture plate and demonstrated typical spindle shape following first-passage monolayer culture (FIGS. 3A and 3B). MSC-derived chondrogenic and osteogenic cells, after encapsulation in bilayered PEG-based hydrogels followed by 6-week incubation separately in either chondrogenic or osteogenic media, resided in their respective layers of the osteochondral construct without crossing the interface (FIG. 3C), corroborating the in vivo findings of layer-specific localization of MSC-derived chondrogenic and osteogenic cells (cf., FIG. 2A). The majority of encapsulated cells remained viable after photoencapsulation as demonstrated by fluorescent live-dead cell staining (live cells labeled green with calcein) (FIG. 3D).

### EXAMPLE 3

### Nanomechanical Properties of Chondrogenic and Osteogenic Constructs

MSC-derived chondrogenic and osteogenic cells encapsulated in PEG hydrogel constructs were separately incubated in chondrogenic or osteogenic medium for 4 weeks and then subjected to nanoindentation with atomic force microscopy (AFM). Three typical force-volume curves for PEG hydrogel (FIG. 3E), PEG hydrogel with MSC-derived chondrogenic cells (FIG. 3F), and PEG hydrogel with MSC-derived osteogenic cells (FIG. 3G) demonstrated different nanoindentation forces upon both approaching and retracting phases of the AFM scanning tip.

Chondrogenic and osteogenic constructs showed significantly different Young's moduli (FIG. 3H), which are defined as the slope of the strain vs. stress curve and represent the elastic mechanical properties of the material under study. The average Young's modulus of osteogenic constructs was 582±59 Kilopascal (kPa), significantly higher than chondral constructs (329±54 kPa), which in turn were significantly higher than PEG hydrogel alone (166±23 kPa) (P<0.01) (FIG. 3H). These nanomechanical data suggest that MSC-derived osteogenic cells encapsulated in PEG hydrogel have produced stiffer matrices than matrices synthesized by MSC-derived chondrogenic cells, both of which are significantly stiffer than PEG hydrogel alone (FIG. 3H).

### EXAMPLE 4

### MSC-Driven Chondrogenesis In PEG Hydrogel Ex Vivo

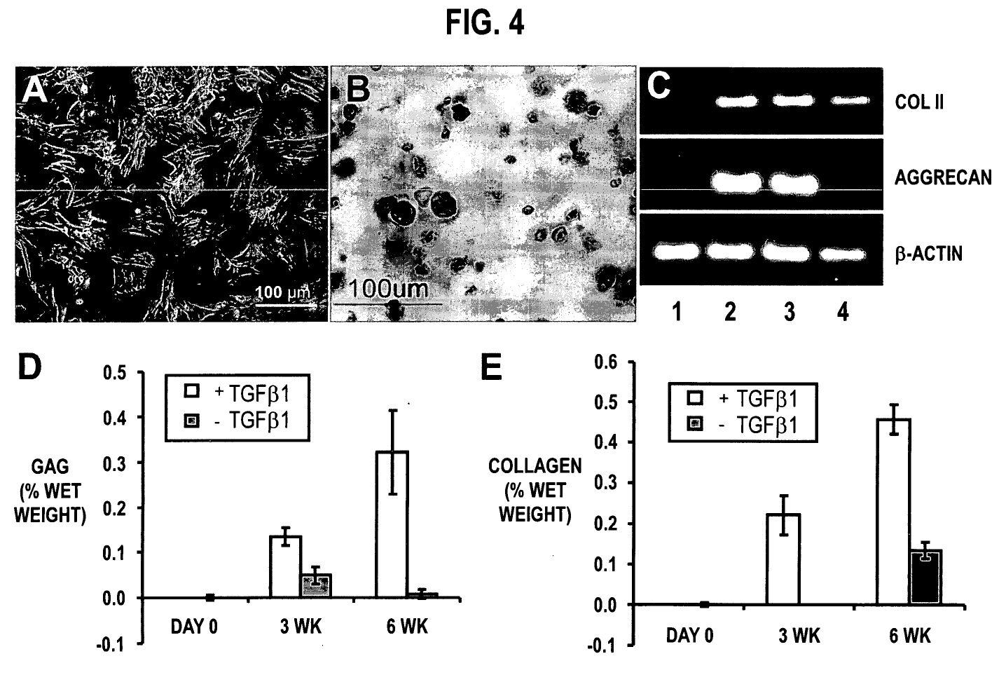

MSCs induced to differentiate into chondrogenic cells after 4-week monolayer culture in TGF-β1-containing chondrogenic medium showed intense reaction to safranin O (FIG. 4A), representing synthesis of cartilage-specific glycosaminoglycans (GAG). After photoencapsulation in PEG-based hydrogel, MSC-derived chondrogenic cells continued to show intense safranin O reaction, especially in their pericellular matrix (FIG. 4B). RT-PCR data corroborated histological findings by showing the expression of aggrecan and type II collagen genes after 6-week incubation in chondrogenic medium (FIG. 4C). PEG hydrogel encapsulating MSC-derived chondrogenic cells showed significant increases in GAG content and total collagen content (% ww) by detection of chondroitin sulfate and hydroxyproline respectively following zero, 3 and 6 weeks of incubation in chondrogenic medium (FIGS. 4D and 4E respectively).

### EXAMPLE 5

### MSC-Driven Osteogenesis In PEG Hydrogel Ex Vivo

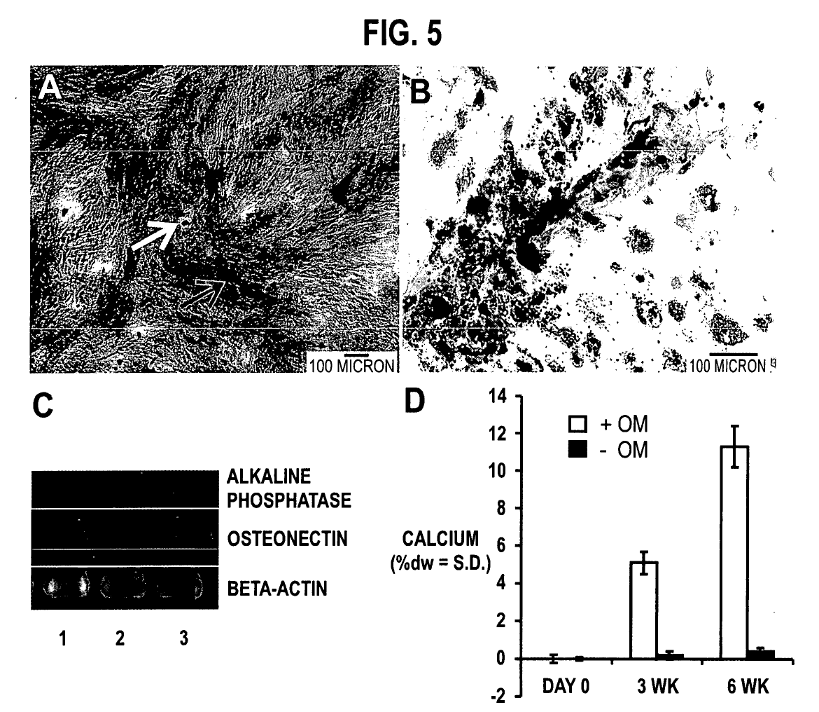

Monolayer MSCs cultured 4 weeks in osteogenic medium containing dexamethasone, β-glycerophosphate, and ascorbic acid exhibited mineral deposits (lower arrow in FIG. 5A) and positive reaction to alkaline phosphatase (upper arrow FIG. 5A). MSC-derived osteogenic cells encapsulated in PEG-hydrogel incubated 4 weeks in osteogenic medium reacted positively to von Kossa stain and contained mineral nodules (FIG. 5B), and expressed osteonectin and alkaline phosphatase genes by RT-PCR analysis (FIG. 5C). A quantitative calcium assay revealed large increases in calcium content in MSC-derived osteogenic constructs as a function of incubation time in osteogenic medium from 0 to 6 weeks (FIG. 5D).

Experimental Protocol

A. Isolation of Marrow-Derived Mesenchymal Stem Cells

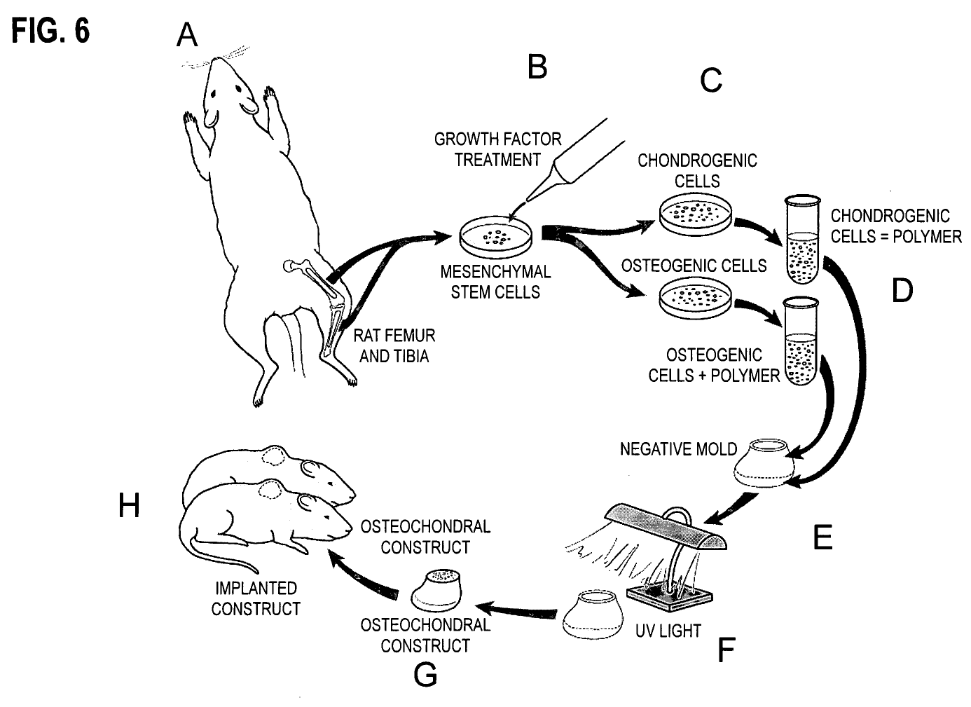

Rat bone marrow-derived MSCs were harvested from 2-4 month-old (200-250 g) male Sprague-Dawley rats (FIG. 6A) (Harlan, Indianapolis, Ind.). Following CO₂ asphyxiation, the tibia and femur were dissected. Whole bone marrow plugs were flushed out with a 10-ml syringe filled with Dulbecco's Modified Eagle's Medium-Low Glucose (DMEM-LG; Sigma, St. Louis, Mo.) supplemented with 10 percent fetal bovine serum (FBS) (Biocell, Rancho Dominguez, Calif.) and 1% antibiotic-antimycotic (Gibco, Invitrogen, Carlsbad, Calif.).

Marrow samples were collected and mechanically disrupted by passage through 16-, 18-, and 20-gauge needles (FIG. 6B). Cells were centrifuged, resuspended in serum-supplemented medium, counted and plated at 5×10⁷ cells/100-mm culture dish and incubated in 95% air/5% CO₂ at 37° C., with fresh medium change every 3-4 days. Upon reaching near confluence, primary MSCs were trypsinized, counted, and passaged at a density 5-7×10⁵ cells/100-mm dish.

In separate studies, the femoral bone marrow content of approximately 3-year-old, castrated male goats was aspirated into 10 ml syringes. Marrow samples were washed and centrifuged twice (1000 rpm for 10 minutes) in mesenchymal stem cell growth media (BioWhittaker, Walkersville, Md.). Cells were counted and plated in 75 cm² flasks at a density of approximately 12,000 cells/cm².

The first medium change occurred after four days, and then media were changed every two to three days until the cells were near confluency. Cells were passaged with 0.025% Trypsin/EDTA (BioWhittaker, Walkersville, Md.) for five minutes at 37° C. and replated in 75 cm² or 175 cm² flasks at 5,000 cells/cm². All animal studies received appropriate approval from the University of Illinois at Chicago and Johns Hopkins University.

B. Hydrogel/Photoinitiator Preparation

Poly(ethylene glycol) diacrylate (PEGDA) (Shearwater, Huntsville, Ala.) was dissolved in sterile PBS supplemented with 100 units/ml penicillin and 100 mg/ml streptomycin (Gibco, Invitrogen, Carlsbad, Calif.) to a final solution concentration of 10% w/v. A photoinitiator, 2-hydroxy-1-[4-(hydroxyethoxy) phenyl]-2-methyl-1-propanone (Ciba, Tarrytown, N.Y.), was added to the PEGDA solution (0.05% w/v).

C. Ex Vivo MSC Differentiation and Cell-PEG Hydrogel Incubation

A single population of first-passage MSCs was cultured separately in chondrogenic or osteogenic medium. Chondrogenic medium contained 10 ng/ml TGF-β1 (RDI, Flanders, N.J.) and 100 U penicillin/100 μg/ml streptomycin (Gibco), whereas osteogenic medium contained 100 nM dexamethasone, 10 mM β-glycerophosphate, and 0.05 mM ascorbic acid-2-phosphate (Sigma) with 100 U penicillin/100 μg/ml streptomycin (Gibco) (FIG. 6C). Cultures were incubated in 95% air/5% CO₂ at 37° C. with medium changes every 3-4 days.

D. MSC-Hydrogel Construct Fabrication For In Vivo Implantation

Upon reaching near confluence, first-passage MSCs were trypsinized, counted, and resuspended in the polymer/photoinitiator solution at the concentration of about 5×10⁶ cells/ml (FIG. 6D). A 200 μl aliquot of cell/polymer suspension with MSC-derived chondrogenic cells was loaded into condyle-shaped polyurethane negative molds (FIG. 6E). The chondrogenic layer was photopolymerized by a long-wave, 365 nm ultraviolet lamp (Glowmark, Upper Saddle River, N.J.) at an intensity of about 4 mW/cm² for 5 min (FIG. 6F).

A cell/polymer suspension containing MSC-derived osteogenic cells was then loaded to occupy the remainder of the mold, followed by photopolymerization (FIGS. 6E and F). The polymerized osteochondral constructs (FIG. 6G) were removed from the mold, and implanted in subcutaneous pockets in the dorsum of severe combined immunodeficient mice (Harlan, Indianapolis, Ind.).

E. Histology and Biochemical Analysis

Following 4 weeks of subcutaneous implantation, recovered articular condyles were fixed in 10% formalin overnight, embedded in paraffin, and sectioned parallel to the construct's long axis at 5 μm thickness. Sequential sections were stained with hematoxylin and eosin, toluidine blue, von Kossa, and safranin-O/fast green to distinguish osseous and cartilaginous tissues. For biochemical analysis, wet weights (ww) and dry weights (dw) of chondrogenic and osteogenic constructs (N=3-4 each) after in vitro incubation were obtained after 48 hours of lyophilization. The dried constructs were crushed and digested in 1 ml of papainase (1.25 μg/ml papain, Worthington, Lakewood, N.J.), 100 mM PBS, 10 mM cysteine, and 10 mM EDTA (pH 6.3) for 18 hours at 60° C. DNA content (ng of DNA/mg dw of the hydrogel) was determined using Hoechst 33258 machine. Glycosaminoglycan (GAG) content was determined using dimethylmethylene blue dye. Total collagen content was determined by measuring the hydroxyproline content after acid hydrolysis and reaction with p-dimethylaminobenzaldehyde and chlorimine-T using 0.1 as the ratio of hydroxyproline to collagen. Calcium content was measured using Sigma Kit 587 (N=3-4). Statistical significance was determined by ANOVA and post-hoc Bonferroni test at an alpha level of 0.05.

F. RNA Extraction and RT-PCR (Reverse Transcription Polymerase Chain Reaction)

Total RNA was isolated from chondrogenic or osteogenic constructs using a RNeasy Kit (Qiagen, Valencia, Calif.). The constructs were homogenized (Pellet Pestle Mixer; Kimble/Kontes, Vineland, N.J.) in 1.5 ml microcentrifuge tubes containing 200 μl of RLT buffer. Then, 400 μl RLT buffer was added, followed by further homogenization with the QIAshredder™ (Qiagen) column. The homogenates were transferred to columns after addition of an equal volume of 70% ethanol.

The RNA was reverse-transcribed into cDNA using random hexamers with the superscript amplification system (Gibco). One-microliter aliquots of the resulting cDNA were amplified in 50 μl volume at annealing temperature of 58° C. (collagen type II was annealed at 60° C.) for 35 cycles using the Ex Taq DNA Polymerase Premix (Takara Bio, Otsu, Shiga, Japan).

PCR primers (forwards and backwards, 5′ to 3′) were as follows:

| | |
|---|---|
| collagen II: | |
| 5′-GTGGAGCAGCAAGAGCAAGGA-3′, | SEQ ID NO:1 |
| and | |
| 5′-CTTGCCCCACTTACCAGTGTG-3′; | SEQ ID NO:2 |
| aggrecan: | |
| 5′-CACGCTACACCCTGGACTTG-3′, | SEQ ID NO:3 |
| and | |
| 5′-CCATCTCCTCAGCGAAGCAGT-3′; | SEQ ID NO:4 |
| β-actin: | |
| 5′-TGGCACCACACCTTCTACAATGAGC-3′, | SEQ ID NO:5 |
| and | |
| 5′-GCACAGCTTCTCCTTAATGTCACGC-3′; | SEQ ID NO:6 |
| osteonectin | |
| 5′-ACGTGGCTAAGAATGTCATC-3′, | SEQ ID NO:7 |
| and | |
| 5′-CTGGTAGGCGA-3′; | SEQ ID NO:8 |
| and | |
| alkaline phosphatase: | |
| 5′-ATGAGGGCCTGGATCTTCTT-3′, | SEQ ID NO:9 |
| and | |
| 5′-GCTTCTGCTTCTGAGTCAGA-3′. | SEQ ID NO:10 |

Each PCR product was analyzed by separating 4 μl of the amplicon and 1 μl of loading buffer in a 2% agarose gel in TAE buffer. Relative band intensities of the genes of interest were compared to those of the housekeeping gene.

G. Nanoindentation with Atomic Force Microscopy

MSC-derived chondrogenic and osteogenic cells encapsulated in photopolymerized PEG hydrogel constructs were separately incubated in chondrogenic or osteogenic media respectively for 4 weeks and then subjected to nanoindentation with Nanoscope IIIa atomic force microscope (AFM) (Veeco-Digital Instruments, Santa Barbara, Calif.). PEG hydrogel incubated in DMEM served as controls. All constructs were prepared in approximately 3×3×3 mm blocks. Force spectroscopy images were obtained in contact mode using AFM fluid chamber by driving the cantilever tip in the Z plane. Cantilevers with a nominal force constant of k=0.12 N/m and oxide-sharpened Si₃N₄ tips were used to apply nanoindentation against the construct's surface. Scan rates and scan size were set at 14 Hz and 50 μm², respectively. Force mapping involved data acquisition of nanoindentation load and corresponding displacement in the Z plane during both extension and retraction of the cantilever tip.

Young's modulus (E) was calculated from force spectroscopy data using the Hertz model, which defines a relationship between contact radius, the nanoindentation load, and the central displacement: where E is the Young's modulus, F is the applied nanomechanical load, v is the Poisson's ratio for a given region, R is the radius of curvature of the AFM tip, and δ is the amount of indentation. Differences in average Young's moduli among PEG hydrogel alone, PEG hydrogels encapsulating MSC-derived chondrogenic and osteogenic cells were detected by ANOVA and post-hoc Bonferroni test at an alpha level of 0.05.

$$E = \frac{3 F ( 1 - {v}^{2} )}{4 \sqrt{R} {δ}^{\frac{3}{2}}}$$

From the foregoing, it will be observed that numerous modifications and variations can be effected without departing from the true spirit and scope of the present invention. It is to be understood that no limitation with respect to the specific examples presented is intended nor should be inferred. The disclosure is intended to cover by the appended claims modifications as fall within the scope of the claims. Each of the patents and articles cited herein is incorporated by reference. The use of the article “a” or “an” is intended to include one or more.

1. A joint or portion thereof prepared de novo by growing stem cells on a biocompatible scaffold.

2. The joint of claim 1 wherein stem cells are derived from bone marrow cells, adipose tissue or peripheral blood.

3. The joint of claim 1 prepared in vivo.

4. The joint of claim 1 prepared ex vivo.

5. A partial or entire joint in human form prepared in vivo or ex vivo by growing stem cells on a biocompatible scaffold comprised of polymerized (polyethylene glycol) diacrylate or other biocompatible polymers.

6. An osteochondral construct from which a joint is fabricated comprising a biocompatible scaffold and stem cells.

7. The construct of claim 6 wherein the stem cells are embryonic or adult mesenchymal stem cells obtained from bone marrow, adipose tissue or peripheral blood.

8. The construct of claim 7 wherein the stem cells are differentiated into chondrocyte and osteoblast cells.

9. The construct of claim 7 wherein the scaffold is in a physical form selected from the group consisting of solid, liquid, gel, mesh, powder, sponge, and paste.

10. The construct of claim 9 wherein the scaffold comprises a hydrogel polymer.

11. The construct of claim 10 wherein the hydrogel polymer is polymerized (polyethylene glycol) diacrylate.

12. The construct of claim 7 wherein the scaffold comprises a polymer selected from the group consisting of polylactic acid, polyglycolic acid, polymerized (polyethylene glycol) diacrylate, polymerized (polyethylene glycol) dimethacrylate and mixtures thereof.

13. The construct of claim 7 wherein the scaffold comprises a material selected from the group consisting of alginate, chitosan, coral, agarose, fibrin, collagen, bone, silicone, cartilage, hydroxyapatite, calcium phosphate, and mixtures thereof.

14. The construct of claim 7 further comprising an osteogenic agent.

15. The construct of claim 14 wherein the osteogenic agent is dexamethasone, member of the bone morphogenetic protein or transforming growth factor families.

16. The construct of claim 7 further comprising a chondrogenic agent.

17. The construct of claim 16 wherein the chondrogenic agent is selected from the group consisting of a glucocorticoid, a member of the transforming growth factor-beta super family, a vitamin A analog and mixtures thereof.

18. A composition in the shape of a partial or entire joint comprising:
(a) a biocompatible scaffold comprised of a scaffold, an osteogenic agent, a chondrogenic agent, a nutrient medium and at least one antibiotic; and
(b) stem cells.

19. The composition of claim 18 wherein the stem cells are adult mesenchymal stem cells.

20. The composition of claim 18 wherein the matrix comprises polymerized (polyethylene glycol) diacrylate.

21. The composition of claim 18 wherein the osteogenic agent is dexamethasone.

22. The composition of claim 18 wherein the chondrogenic agent is selected from the group consisting of a glucocorticoid, a member of the transforming growth factor-beta super family, a vitamin A analog and mixtures thereof.

23. The composition of claim 18 wherein the biocompatible scaffold is comprised of polymerized (polyethylene glycol) diacrylate, dexamethasone, transforming growth factor beta-1, a nutrient medium comprising beta-glycerophosphate and ascorbic acid 2-phosphate, penicillin, and streptomycin.

24. The composition of claim 23 wherein at least some the stem cells are differentiated into a chondrocyte and an osteoblast.

25. A method of producing an osteochondral construct comprising the steps:
(a) providing stem cells;
(b) treating one portion of the cells with chondrogenic medium to induce differentiation into chondrocytes;
(c) treating a second portion of the cells with osteogenic medium to induce differentiation into osteoblasts; and
(d) loading the chondrocytes and osteoblasts onto a biocompatible scaffold.

26. The method of claim 25 wherein the stem cells are adult mesenchymal stem cells from bone marrow.

27. A method of producing a biologically engineered partial or entire joint in vivo comprising implanting a composition comprising a biocompatible scaffold and stem cells into a host.

28. A method of producing a biologically engineered partial or entire joint ex vivo comprising admixing stem cells, an osteogenic agent, a chondrogenic agent, a nutrient medium and at least one antibiotic with a biocompatible scaffold that is comprised of a matrix.

29. The method of claim 28 further comprising subjecting the cells to mechanical stresses conducive to either osteogenesis or chondrogenesis or both.

30. A method of producing a biologically engineered partial or entire joint in vivo comprising the steps:
(a) providing adult mesenchymal stem cells (MSCs) from bone marrow;
(b) expanding the MSCs;
(c) treating a first portion of the expanded MSCs with chondrogenic medium containing TGF-β1;
(d) treating a second portion of the expanded MSCs with osteogenic medium containing dexamethasone, β-glycerophosphate, and ascorbic acid;
(e) forming a PEG-hydrogel monomer suspension of the MSC-derived chondrogenic cells;
(f) forming a PEG-hydrogel monomer suspension of the MSC-derived osteogenic cells;
(g) loading the PEG-hydrogel monomer suspension of MSC-derived chondrogenic cells in a negative mold of a joint or partial joint;
(h) loading the PEG-hydrogel monomer suspension of MSC-derived osteogenic cells in the negative mold of the joint or partial joint;
(i) photopolymerizing the PEG-hydrogel monomer suspensions with UV light to form a fabricated osteochondral construct;
(j) implanting the fabricated osteochondral construct in a host;
(k) maintaining the host with the implant for a time period sufficient for the osteochondral construct to form a joint or partial joint; and
(l) harvesting a joint or partial joint prepared from the osteochondral construct.
